(19) United States
(12) Patent Application Publication
(10) Pub. No.: US 2022/0347141 A1
(43) Pub. Date: Nov. 3, 2022

(54) METHODS OF TREATING PHOSPHATE CONCENTRATION DISORDERS WITH L-BAIBA

(71) Applicants: YALE UNIVERSITY, New Haven, CT (US); THE TRUSTEES OF INDIANA UNIVERSITY, Bloomington, IN (US); BOARD OF REGENTS, THE UNIVERSITY OF TEXAS SYSTEM, Austin, TX (US)

(72) Inventors: Clemens BERGWITZ, New Haven, CT (US); Lynda BONEWALD, Indianapolis, IN (US); Marco BROTTO, Austin, TX (US)

(21) Appl. No.: 17/761,020

(22) PCT Filed: Sep. 17, 2020
(86) PCT No.: PCT/US2020/051212

§ 371 Date: Mar. 16, 2022

Related U.S. Application Data
(60) Provisional application No. 62/901,616, filed on Sep. 17, 2019.

(51) Int. Cl. A61K 31/197; A61P 3/12; A61K 45/06

(52) U.S. Cl. CPC A61K 31/197; A61P 3/12; A61K 45/06

(57) ABSTRACT

Provided herein are methods and compositions for treating nephropathic conditions such as chronic kidney disease, as well as phosphate concentration disorders and myopathy related to phosphate concentration disorders. The methods and compositions include administering a therapeutically effective amount of L-BAIBA ((S)-β-aminoisobutyric acid) to a subject in need thereof.

### CROSS-REFERENCE TO RELATED APPLICATION

This application claims priority to U.S. Provisional Patent Application Ser. No. 62/901,616 entitled “METHODS OF TREATING PHOSPHATE CONCENTRATION DISORDERS WITH L-BAIBA,” filed Sep. 17, 2019, the disclosure of which is incorporated herein by reference in its entirety.

### STATEMENT REGARDING FEDERALLY SPONSORED RESEARCH

This invention was made with government support under AG039355, DK079310, and AR070717 awarded by the National Institutes of Health. The government has certain rights in the invention.

### BACKGROUND

Phosphorus is one of the most abundant elements in the body, and is primarily found in the form of phosphates in bone, although soft tissue contains phosphate conjugates such as nucleic acids and phospholipids. Between 80-90% of the phosphate in the body is contained in bone, where it is responsible for helping maintain the strength and structure of the skeleton. Because of its importance to the structural integrity of the body, and its crucial role in the energy storage molecules ADP and ATP, maintaining phosphate homeostasis is of vital importance.

Skeletal muscle also requires inorganic phosphate (Pi) for energy storage, signal transduction, and acid-base balance. However, the bulk of Pi as substrate, which is needed in large quantities for ATP synthesis in skeletal muscle, may be recycled endogenously following ATP hydrolysis, rather than supplied from EC Pi in the circulation. Pi is important for muscle function as a signal in addition to serving as substrate for muscle metabolism. Since the principal reservoir for Pi is not muscle but the skeleton both tissues must communicate, which we here show involves the actions of the myokine L-BAIBA. Pi transporter deficient muscle secretes the myokine L-BAIBA to modify bone and mineral metabolism, by stimulating secretion of the osteocyte factor Fibroblast growth factor 23 (FGF23) which in turn stimulates renal Pi excretion and lowers blood Pi. L-BAIBA also protects osteocytes from apoptosis and as such improves bone strength.

Rapid lowering of blood Pi profoundly impairs muscle function and can lead to significant clinical problems. Respiratory compromise, and even heart failure are well-recognized complications of low blood Pi in intensive care settings. Low blood Pi also complicates nutritional and genetic forms of rickets and is thought to be a major contributor to the muscle weakness and early fatigue in the vitamin D-, and therefore Pi-deficient, elderly population. Npt2a knockout mice (Npt2a^{-/-}), a model for the human renal Pi wasting condition hereditary hypophosphatemic rickets with hypercalciuria (HHRH), have reduced blood Pi due to renal Pi wasting, and these animals have impaired myocyte mitochondrial ATP synthesis (V_{ATP}) and reduced muscle function. Since L-BAIBA improves muscle function in an autocrine fashion, secretion of L-BAIBA by Pi transporter deficient muscle may be a compensatory response.

The mouse models demonstrate that smPit1^{-/-} and smPit2^{-/-} mice have impaired muscle function that resembles hypophosphatemic myopathy observed in Npt2a^{-/-} mice. Several compensatory mechanisms on the cellular level (down regulation of Xpr1), tissue levels (paracrine L-BAIBA) and systemic level (FGF23-independent renal Pi wasting) were observed and suggests that muscle modifies mineral metabolism. Regulation of L-BAIBA further supports the unexpected discovery that Pit1 and/or Pit2 function as endocrine regulators in addition to serving as transporter for Pi as a substrate.

Low blood Pi causes bone loss and clinically significant myopathy. Mice with skeletal muscle (sm)-specific deletion of both alleles of the type III Pi transporters Pit1 (Slc20a1) and Pit2 (Slc20a1) (DKO) is perinatally lethal due to a severe myopathy causing skeletal muscle degeneration. Mice with expression of one or two transporter alleles display a reduction in running endurance, while different from findings in Hyp—a murine model of X-linked hypophosphatemia—grip strength is unaffected, suggesting that FGF23 excess contributes to the myopathy seen in XLH. This may be because iFGF23 is low normal in these mice and/or because FGF23 signaling is modified by Pit1 and Pit2 in skeletal muscle. Metabolic analysis shows that DKO and smPit1^{-/+}; smPit2^{-/-} mice have an FGF23-independent renal phosphate leak and a screen for skeletal muscle derived factors identified revealed elevated circulating levels of the myokine L-BAIBA in these mice. L-BAIBA is a mediator of the beneficial effect of exercise from skeletal muscle to other organs in an endocrine manner and it reduces inflammation in skeletal muscle in an autocrine/paracrine manner.

Chronic hypophosphatemia is more commonly caused by vitamin D deficiency and less commonly by acquired or inherited Pi-wasting disorders. The mechanism of hypophosphatemic myopathy is poorly understood, but clues provided by phosphate related disorders suggest that it is not simply caused by lack of intracellular (IC) Pi as a substrate for the maintenance of muscle oxidative metabolism. During refeeding, for example, hypophosphatemia is caused by increased cellular uptake of Pi, and therefore muscle IC Pi is high, not low, suggesting that low blood and extracellular Pi (EC Pi) are a key determinant of muscle function, in addition to IC Pi which is an important substrate for ATP synthesis. Myopathy in X-linked hypophosphatemia (XLH) develops in the setting of high circulating levels of fibroblast growth factor 23 (FGF23), low 1,25(OH)₂-vitamin D (1,25D), and elevated parathyroid hormone (PTH), while myopathy in hereditary hypophosphatemic rickets with hypercalciuria (HHRH) develops in the setting of suppressed FGF23 and elevated 1,25D resulting in suppressed PTH.

These data argue against causal roles of these hormones in myopathy, although some reports suggest that they may also contribute to muscle function. FGF23, for example, appears to be also responsible for functional impairment of cardiac muscle, hypertrophy, fibrosis and reduced lifespan of individuals with CKD and individuals treated with burosumab (Crysvita), an FGF23-neutralizing antibody therapy for XLH, uniformly report improved fatigue and endurance. Like patients with XLH, Hyp mice develop rickets/osteomalacia due to FGF23-dependent hypophosphatemia and have reduced grip strength and running wheel activity, which normalizes after therapy with anti-FGF23 antibody therapy, which restores blood Pi in these animals.

Similar to Hyp mice, the Dmp1 null mouse model (Dmp1^{-/-}) has hypophosphatemia due to elevated plasma FGF23. Ex vivo functional testing shows reduced force of the Dmp1^{-/-} extensor digitorum longus (EDL-fast-twitch muscle) and soleus (SOL-slow-twitch muscle) muscles, arguing for structural changes that are induced by chronic exposure to hypophosphatemia and/or elevated FGF23 that can be detected despite normal Pi levels in the ex vivo muscle culture setting. Since direct administration of FGF23 does not influence skeletal muscle cell proliferation and differentiation or ex vivo muscle contractility, other endogenous substances may be required to act in concert with FGF23 or apart from FGF23 to promote muscle dysfunction in hereditary hypophosphatemic rickets and CKD. Furthermore, skeletal muscle lacks expression of the co-receptor klotho (KL) and it is unclear, how FGF23 may activate FGFR1 or other receptors in this tissue. One possibility is that Pit1 and/or Pit2 substitute for KL in skeletal muscle, since there is in vitro evidence that Pi can facilitate FGF-signaling which may involve Pit1 and Pit2.

There are several roles for Pi-transporters in muscle function: the skeletal muscle-specific double knockout (smPit1^{-/-}; smPit2^{-/-}), which avoids embryonic lethality or systemic changes seen in the global knockouts and to exclude homeostatic changes, is perinatally lethal due to severe muscle weakness and failure to thrive. The ablation of one or two alleles of Pit1 and/or Pit2 results in a gene-dose-dependent reduction of running endurance (smPit2^{-/-}>smPit1^{-/-}>smPit1^{-/+}; smPit2^{-/+}>smPit1^{-/-}; smPit2^{-/+} or smPit1^{-/+}; smPit2^{-/-}), while different from findings in Hyp—a murine model of X-linked hypophosphatemia—grip strength is unaffected, suggesting that FGF23 excess contributes to the myopathy seen in XLH. Evaluation of these mouse lines showed presence of compensatory mechanisms on the cellular level, tissue levels and systemic level which suggest muscle-bone and muscle-kidney interaction. In compensation for loss of Pit1 in skeletal muscle the Pi-exporter Xenotropic and Polytropic Retrovirus Receptor 1 (Xpr1) mRNA is down-regulated in in smPit1^{-/-} mice, which, without being bound by theory, compensates for reduced sensing of EC Pi and high dietary Pi intake restores muscle function and Xpr1 expression in Npt2a^{-/-} mice, but not in smPit1^{-/-} mice.

On the tissue levels increased secretion of L-BAIBA was identified. L-BAIBA is the S-enantiomer of beta amino-isobutyric acid produced from the utilization of a L-valine as an energy source under the control of the mitochondrial transcriptional co-activator PGC-1a. L-BAIBA is detected at levels of 0.8 μM in human serum at a 1:4 D:L ratio. It was shown to be a mediator of the beneficial effect of exercise from skeletal muscle to other organs in an endocrine manner and stimulates hepatic fatty acid oxidation, the browning of white adipose tissue, insulin sensitivity, it reduces inflammation in skeletal muscle in an autocrine/paracrine manner and protects osteocytes from cell death due to oxidative.

L-BAIBA is also thought to act on young osteocytes which express high levels of MRGPRD, Mas-related G protein-coupled receptor type D, the receptor for L-BAIBA, while older osteocytes do not. L-BAIBA activation of MRGPRD in young mice promotes osteocyte survival by maintaining mitochondrial integrity, which in turn enhances bone formation by osteoblasts and prevents bone loss caused by hind-limb suspension. The low levels of MRGPRD expression in older osteocytes impair the capacity of L-BAIBA to preserve mitochondrial integrity under oxidative stress, contributing to loss of osteocytes with age.

In addition, FGF23-independent Pi-excretion in the kidneys of DKO and three allele mutant mice was observed. Unexpectedly, L-BAIBA supplementation of WT mice resulted in increased renal Pi-excretion, which, without being bound by theory, indicates that L-BAIBA has a direct or indirect role in control of phosphate homeostasis.

Pit1 and Pit2 are essential for skeletal muscle function, but this is at least to some degree independent of Pi-uptake since the bulk of Pi as substrate, which is needed in large quantities for ATP synthesis in this organ, may be recycled endogenously following ATP hydrolysis, rather than supplied from EC Pi in the circulation. L-BAIBA production by skeletal muscle improves muscle function in an autocrine/paracrine fashion in mouse models, which may be modified by skeletal muscle Pi transporters, while also having systemic effects on phosphate homeostasis.

In addition to generation of smPit1^{-/-}; smPit2^{-/-} mice a transgenic mouse expressing epitope-tagged human PIT1 transporter under control of the CMV/chicken beta actin (CAG) promoter and a loxP-stop-loxP (LSL) cassette was studied. This construct allows Cre-mediated activation of transgene expression. Germline excision of the LSL cassette results in expression of the transgene in all mouse tissues (HA-hPit1^{+/tg}). Recombination was confirmed using genomic DNA obtained from tail samples of these mice. The expression of HA-hPIT1 was found to be approximately 1000-fold above background and 10-fold above endogenous mouse Pit1 in cultured primary calvaria osteoblasts (PCOB) when assessed by Pit1 immunoblot and qRT-PCR. In addition, sodium-dependent ³²Pi-uptake was 1.3-fold higher in HA-hPit1^{+/tg} PCOB cultures than in wildtype (WT) cultures (0.0014±9.89E-05 vs. 0.0011±8.52E-05, p=0.037). HA-hPit1^{+/tg} mice showed 1.3-fold higher plasma Pi levels when compared to WT (10.2±0.89, n=11 vs. 7.8±0.62, n=17, p=0.032), while intact FGF23 and urine Pi excretion index (PEI) were normal.

The mouse models demonstrate that smPit1^{-/-} and smPit2^{-/-} mice have impaired muscle function that resembles hypophosphatemic myopathy observed in Npt2a^{-/-} mice. Several compensatory mechanisms on the cellular level (down regulation of Xpr1), tissue levels (paracrine L-BAIBA) and systemic level (FGF23-independent renal phosphate wasting) were observed and suggests that muscle modifies mineral metabolism. Regulation of L-BAIBA further supports the unexpected discovery that Pit1 and/or Pit2 function as endocrine regulators in addition to serving as transporter for Pi as a substrate.

Chronic kidney disease (CKD) is a gradual and progressive loss of the ability of the kidneys to excrete wastes, concentrate urine, and conserve electrolytes. Unlike acute renal failure with its abrupt but reversible renal function loss, renal functions in chronic renal disease progress and deteriorate irreversibly towards end-stage renal disease. As a result of hyperphosphatemia and chronic inflammation FGF23 is markedly elevated in CKD and can cause left ventricular hypertrophy resulting in heart failure and death. L-BAIBA secreted from skeletal muscle in CKD may contribute to pathophysiology of metabolic bone disease (MBD), left ventricular myopathy and protein wasting myopathy seen in CKD.

Many medications to increase blood Pi in hypophosphatemic disorders such as X-linked hypophosphatemia (XLH) and to lower blood Pi in hyperphosphatemic disorders such as CKD have serious side effects, such as stomach cramps and diarrhea, which can prevent patients from continuing with treatment. In turn, both, patients with XLH and CKD often exhibit myopathy, including mitochondrial myopathy, as a result of Pi imbalances in their serum.

Consequently, there is a need for new medications that are efficacious in controlling Pi and FGF23 levels and/or that can prevent or ameliorate myopathy. The compounds and methods described herein address this pressing need.

### BRIEF SUMMARY OF THE INVENTION

In certain embodiments, a method of treating nephropathy is provided. In other embodiments, the method includes administering to a subject in need thereof a therapeutically effective amount of L-BAIBA, or a salt, solvate, polymorph, prodrug, or N-oxide thereof in yet other embodiments, the administration of L-BAIBA includes administration of a composition that includes L-BAIBA, or a salt, solvate, polymorph, prodrug, or N-oxide thereof, at least one additional therapeutic compound, and at least one pharmaceutically acceptable excipient.

In certain embodiments, a method of treating a phosphate concentration disorder is provided. In other embodiments, the method includes administering to a subject in need thereof a therapeutically effective amount of L-BAIBA, or a salt, solvate, polymorph, prodrug, or N-oxide thereof. In yet other embodiments, it was unexpectedly discovered that L-BAIBA, but not D-BAIBA, can be used to treat any nephropathy and/or phosphate concentration disorder described herein.

### BRIEF DESCRIPTION OF THE FIGURES

The figures illustrate generally, by way of example, but not by way of limitation, various embodiments of the present disclosure.

FIG. 1 illustrates a non-limiting embodiment of the present disclosure.

FIG. 2A illustrates that skeletal muscle selective DKO mice for Pit1 and Pit2 have elevated L-BAIBA (orange) and D-BAIBA (green) levels at age P10. Data are mean±SEM, ****p<0.00002, ***p=0.0002, **p=0.002, *p=0.03 vs. WT. FIG. 2B illustrates that double and three-allele mutant mice have elevated L-BAIBA (orange) and D-BAIBA (green) levels. Data are mean±SEM, ****p<0.00002, ***p=0.0002, **p=0.002, *p=0.03 vs. WT.

FIG. 3A illustrates that skeletal muscle selective DKO for Pit1 and Pit2 has FGF23-independent renal Pi wasting resulting in hypophosphatemia at age P10. We postulated that L- and/or D-BAIBA may be the cause of renal Pi wasting in these mice. Data are mean±SEM, ****p<0.00002, ***p=0.0002, **p=0.002, *p=0.03 vs. WT. FIG. 3B illustrates that while DKO die of severe myopathy by age P13, double and three-allele mutant mice have impaired voluntary wheel running activity but otherwise are healthy and fertile. smPit2^{-/-} and three allele mutant mice display FGF23-dependent renal Pi wasting at age P80. The Pi excretion index (PEI) is highest in smPit1^{+/-}; smPit2^{-/-} mice. Data are mean±SEM, ****p<0.00002, ***p=0.0002, **p=0.002, *p=0.03 vs. WT.

FIGS. 4A-4D illustrate that WT mice exposed to diets with different Pi content have unchanged L-BAIBA levels. Thirty mice are rendered hypophosphatemic and hypophosphaturic after low Pi diet for two weeks. This dietary maneuver also suppresses iFGF23, iPTH and increases 1,25-D levels in these mice (data pending). At day 10 five mice are continued on low Pi diet (LPD, 0.02% Pi), 10 mice are switched to control diet (CO, 0.3% Pi), and 10 mice are switched to high Pi diet (HPD, 1.2% Pi), all diets are egg-white based, calorically matched and otherwise identical and contain 0.6% calcium. 3.1 kcal/mouse/day are provided. Mice were sacrificed at day 30 to collect blood, urine and kidneys. Serum L-AABA (FIG. 4A), L-BAIBA (FIG. 4B), D-BAIBA (FIG. 4C), and GABA (FIG. 4D) are shown. Data are mean±SEM, ****p<0.00002, ***p=0.0002, **p=0.002, *p=0.03 vs. CO.

FIGS. 5A-5D illustrate that L-BAIBA treatment increases urine Pi excretion in WT mice in an FGF23-dependent fashion. FIG. 5A: 5 mo. old males treated with CO, L-BAIBA, or D-BAIBA in the drinking water for 5 days. FIGS. 5B-5D: same 5 mo. old males from FIG. 5A treated for 30 days, following sacrifice in addition to urine Pi/urine creatinine, plasma Pi and plasma intact FGF23 were determined. Data are mean±SEM, ****p<0.00002, ***p=0.0002, **p=0.002, *p=0.03 vs. CO.

FIG. 6 illustrates a regression analysis of L-BAIBA, or D-BAIBA plotted over serum Pi. L- and D-BAIBA correlate positively with serum Pi. Serum was obtained from individuals with hypophosphatemia (hypo) caused by XLH and HPT or individuals with hyperphosphatemia (hyper) due to CKD.

FIGS. 7A-7D illustrate a grouped analysis of the same individuals as in FIG. 5. Data are mean±SEM, ****p<0.00002, ***p=0.0002, **p=0.002, *p=0.03 vs. CO.

FIG. 8 illustrates the absence of detectable L-BAIBA and D-BAIBA in a human myofiber experiment.

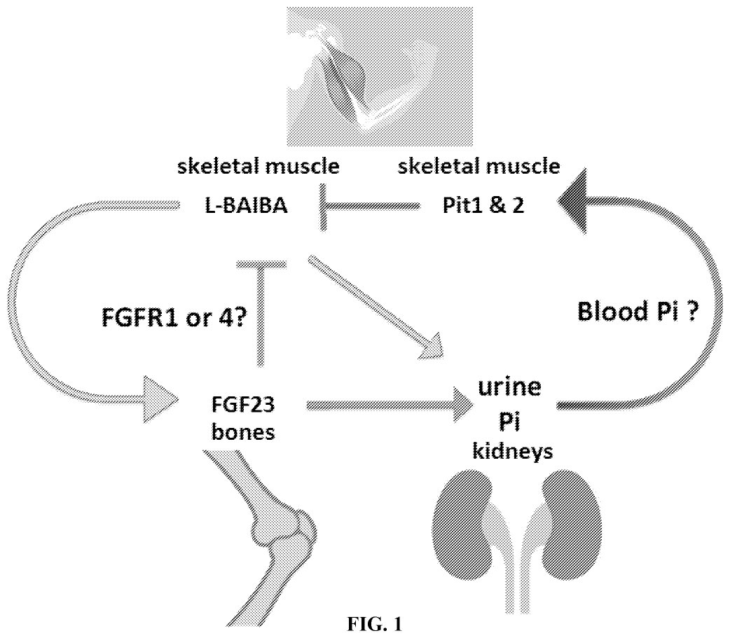

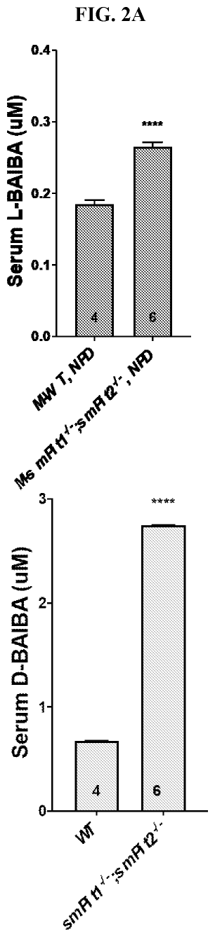

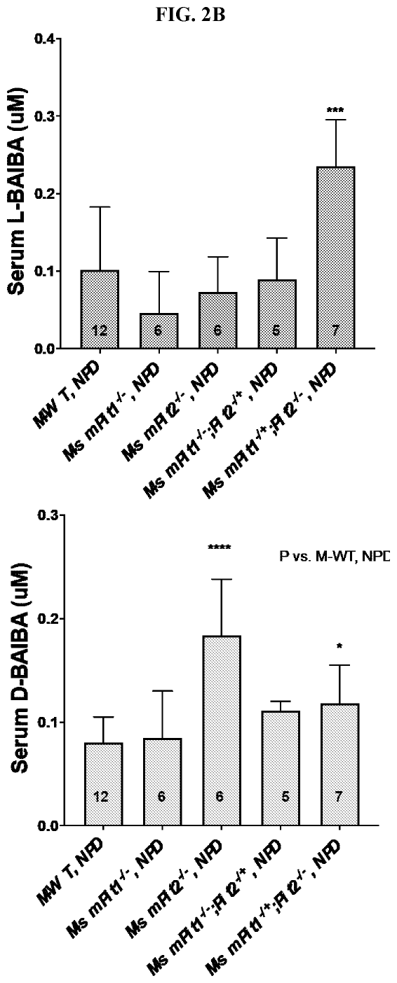

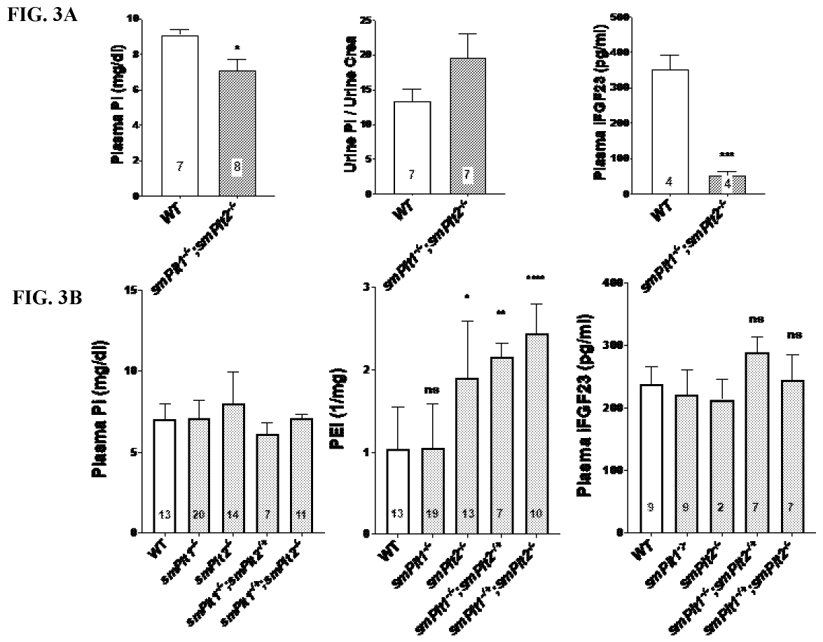

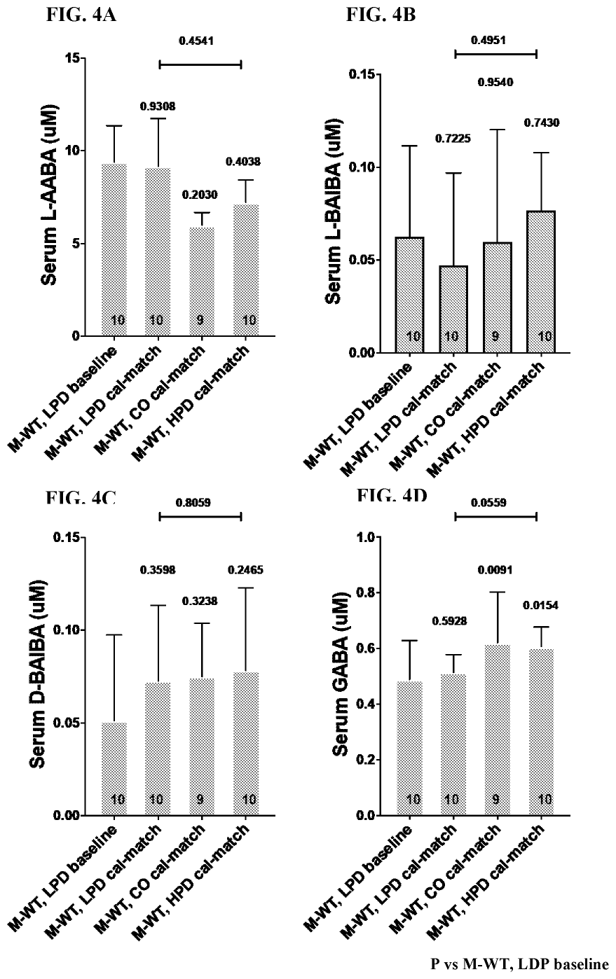

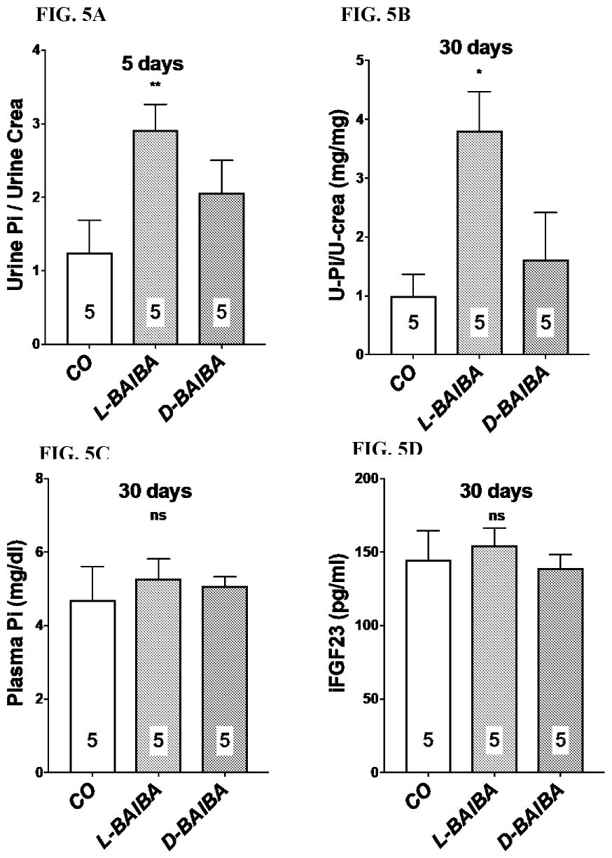

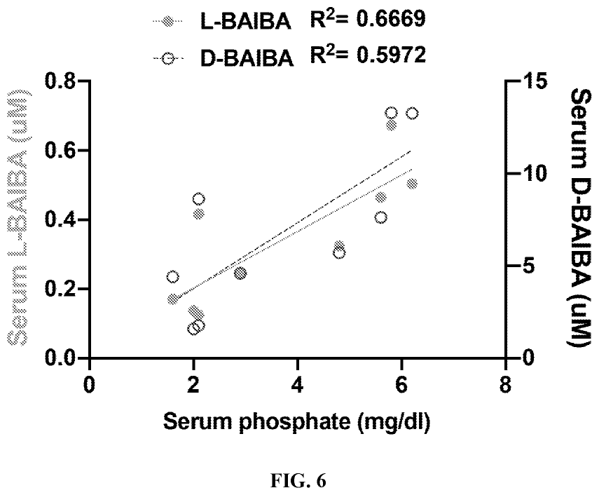

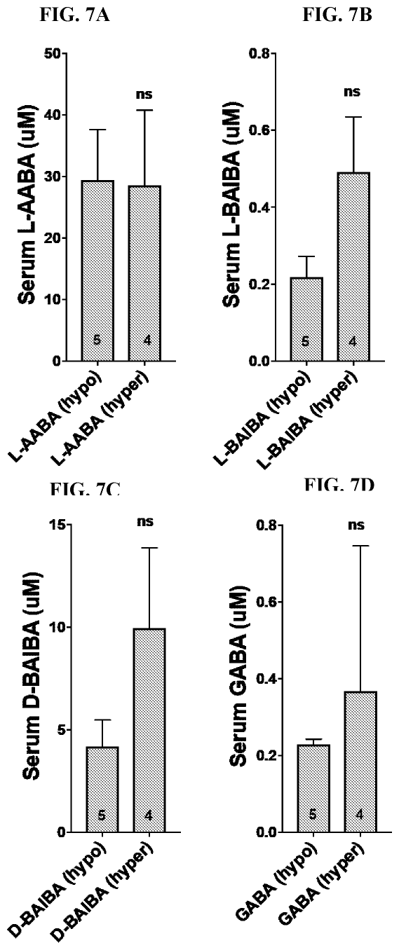

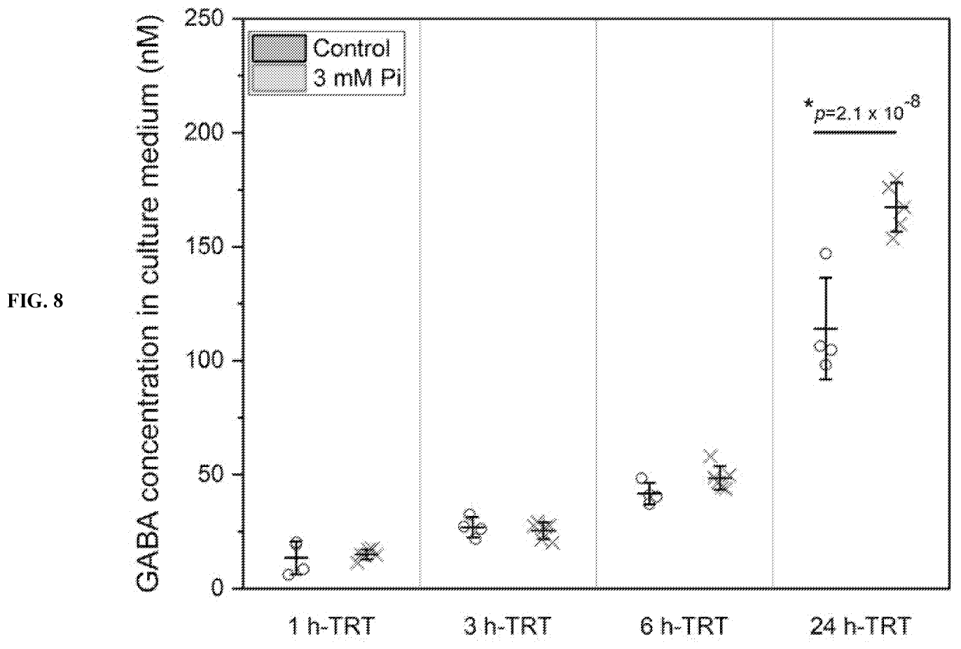

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, Pa.), which is incorporated herein by reference.

As used herein, the term “nephropathy” includes any abnormal condition of the kidney, such as Alport syndrome, diabetic neuropathy, Fabry disease, focal segmental glomeronucleosis, glomerulonephritis, IgA nephropathy (Berger's disease), kidney stones, minimal change disease, nephrotic syndrome, polycystic kidney disease (PKD), and chronic kidney disease (CKD).

As used herein, the term “phosphate concentration disorder” or “phosphate concentration disease” refers to a condition where the subject suffers from hyperphosphatemia or hypophosphatemia. Hyperphosphatemia, where the serum Pi concentration is above highest healthy value of Pi in Table 1, can result from CKD, metabolic acidosis, respiratory acidosis, familial hyperphosphatemic tumoral calcinosis (FHTC), rhabdomyolysis, and/or other conditions that result in abnormally high serum Pi concentrations. Hypophosphatemia, where the serum Pi concentration is below the lowest healthy value of Pi in Table 1, can result from alcoholism, burns, starvation, diuretic use, primary hypoparathyroidism (PHPT), hereditary hypophosphatemic rickets with hypercalciuria (HHRH), X-linked hypophosphatemia (XLH), autosomal dominant hypophosphatemic rickets (ADHR), autosomal recessive hypophosphatemia (ARHP), tumor induced osteomalacia (TIO, also known as oncogenic osteomalacia), and/or other conditions that result in abnormally low serum Pi concentrations.

TABLE 1
Healthy serum Pi concentrations ([Pi])
by age group for males and females.

| Age Range | Male Healthy [Pi] | Female Healthy [Pi] |
|---|---|---|
| (years) | (mg/dL) | (mg/dL) |
| 1-4 | 4.3-5.4 | 4.3-5.4 |
| 5-13 | 3.7-5.4 | 4.0-5.2 |
| 16-17 | 3.1-4.7 | 3.1-4.7 |
| =>18 | 2.5-4.5 | 2.5-4.5 |

In various embodiments, the FHTC is due to loss-of-function mutations in FGF23, KLOTHO or GALNT3. In various embodiments, the HHRH is due to NPT2c and/or NPT2a loss-of-function mutations. In various embodiments, the XLH is due to PHEX mutations. In various embodiments, the ADHR is due to gain-of-function mutations in FGF23. In various embodiments, the ARHP is due to loss-of-function mutations in DMP1 and/or FAM20C. In various embodiments, the TIO is due to FN-FGFR1 and/or FN-FGF1 rearrangements.

As used herein, the term “myopathy” is a disease or disorder of the muscles that results in the improper functioning of the muscle and results in muscular weakness. The myopathy can be result of a variety of disorders, including endocrine, inflammatory, paraneoplastic, infectious, drug- and toxin-induced, critical illness myopathy, metabolic, collagen related, and myopathies with other systemic disorders. In various embodiments, the myopathy is as result of hypophosphatemic disorders selected from PHPT, HHRH, XLH, ADHR, ARHP, or TIO.

As used herein, the term “Pi” refers to inorganic phosphate and any associated counterions if the inorganic phosphate is charged. The inorganic phosphate can be in the form of PO₄³⁻, HPO₄²⁻, H₂PO₄⁻, H₃PO₄, or a combination thereof.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound described herein (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

### Compounds and Compositions

The following examples illustrate non-limiting embodiments of the compositions described herein. In various embodiments, the composition includes L-BAIBA or derivatives thereof. L-BAIBA, and derivatives thereof, can be prepared using synthetic methods known by those skilled in the art. L-BAIBA is also known as (S)-3-amino-2-methylpropanoic acid or (S)-β-aminoisobutyric acid. Derivatives of L-BAIBA include salts, solvates, polymorphs, prodrugs, and N-oxides thereof.

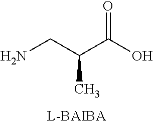

Salts of L-BAIBA include any of the pharmaceutically acceptable organic or inorganic salts described herein. Non-limiting examples of salts of the carboxylic acid in L-BAIBA include ammonium, sodium, potassium, calcium, and lanthanum salts of L-BAIBA, and the like. Non-limiting examples of salts of the amine in L-BAIBA include fluoride, chloride, bromide, acetate, succinate, benzoate, and propionate salts of L-BAIBA, and the like. Solvates of L-BAIBA include hydrates, ½ hydrates, sesquihydrates, and non-aqueous equivalents thereof. Prodrugs of L-BAIBA include any pharmaceutically acceptable prodrugs described herein, including esters, carbamates, carbonates, sulfonamides, phosphate esters, and the like. Prodrugs of L-BAIBA can be formed at the carboxylic acid, the amine, or both.

In various embodiments, L-BAIBA, or a derivative thereof as described herein, can be present in an amount of about 0.001% (w/w) to about 20% (w/w) of a pharmaceutical composition suitable for treatment of any of the diseases or disorders described herein. The L-BAIBA can be present in the pharmaceutical composition in an amount of about 0.001, 0.002, 0.004, 0.006, 0.008, 0.01, 0.02, 0.04, 0.06, 0.08, 0.1, 0.2, 0.4, 0.6, 0.8, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, 10, 11, 12, 13, 14, 15, 16, 17, 18, 19, or 20% (w/w). The compositions of L-BAIBA are, in some embodiments, oral compositions.

The compounds described herein can possess one or more stereocenters, and each stereocenter can exist independently in either the (R) or the (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound disclosed herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds described herein may exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

Compounds described herein are synthesized using any suitable procedures starting from compounds that are available from commercial sources, or are prepared using procedures described herein.

### Pharmacology

In various embodiments, L-BAIBA, or a derivative thereof, can be administered to a subject in an amount ranging from about 0.01 mg/kg to about 200 mg/kg, or about 0.5 mg/kg to about 190 mg/kg, or about 0.75 mg/kg to about 180 mg/kg, or about 1 mg/kg to about 170 mg/kg, or about 1.5 mg/kg to about 160 mg/kg, or about 2 mg/kg to about 150 mg/kg, or about 2.5 mg/kg to about 140 mg/kg, or about 3 mg/kg to about 130 mg/kg, or about 3.5 mg/kg to about 120 mg/kg, or about 4 mg/kg to about 110 mg/kg, or about 4.5 mg/kg to about 100 mg/kg, or about 5 mg/kg to about 95 mg/kg, or about 5.5 mg/kg to about 90 mg/kg, or about 6 mg/kg to about 85 mg/kg, or about 6.5 mg/kg to about 80 mg/kg, or about 7 mg/kg to about 75 mg/kg, or about 7.5 mg/kg to about 70 mg/kg, or about 8 mg/kg to about 65 mg/kg, or about 8.5 mg/kg to about 60 mg/kg, or about 9 mg/kg to about 55 mg/kg or about 9.5 mg/kg to about 50 mg/kg, or about 10 mg/kg to about 45 mg/kg.

In various embodiments, L-BAIBA, or a derivative thereof, can be administered to a subject in an amount that is less than, equal to, or greater than about 0.01 mg/kg, 0.05 mg/kg, 0.1 mg/kg, 0.25 mg/kg, 0.5 mg/kg, 0.75 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 12 mg/kg, 14 mg/kg, 16 mg/kg, 18 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 100 mg/kg, 105 mg/kg, 110 mg/kg, 115 mg/kg, 120 mg/kg, 125 mg/kg, 130 mg/kg, 140 mg/kg, 145 mg/kg, 150 mg/kg, 155 mg/kg, 160 mg/kg, 170 mg/kg, 175 mg/kg, 180 mg/kg, 185 mg/kg, 190 mg/kg, 195 mg/kg, or 200 mg/kg.

### Compositions

The compositions described herein include pharmaceutical compositions containing L-BAIBA, or derivatives thereof, and at least one pharmaceutically acceptable carrier. In certain embodiments, includes a pharmaceutical composition comprising D-BAIBA, or derivatives thereof, and at least one pharmaceutically acceptable carrier. In various embodiments, the pharmaceutical composition contains a racemic mixture of L-BAIBA and D-BAIBA, or derivatives thereof, and at least one pharmaceutically acceptable carrier. The L-BAIBA, in some embodiments, is at least about, or about 95%, 96%, 97%, 98%, 99%, 99.9%, 99.99%, or 99.99% enantiomerically pure L-BAIBA. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

### Methods of Treatment

The therapeutic methods described herein include a method of treating or preventing nephropathy. The Pi metabolism of mice with skeletal muscle selective ablation of Pi transporters Pit1 and Pit2 was evaluated and revealed an FGF23-independent renal Pi leak at age 10 days. A screen for known myokines that could be responsible for this renal Pi leak showed increased serum levels of L-BAIBA. Interestingly, elevate L-BAIBA was also seen in mice lacking three Pi transporter alleles (smPit1^{-/-}; smPit2^{-/+} or smPit1^{-/+}; smPit2^{-/-}). FGF23 levels were inappropriately normal in these mice at age 80 days. 5 month old wild-type (WT) mice were treated with L-BAIBA, which caused a renal Pi leak, consistent with the hypothesis that secretion of L-BAIBA is the myokine responsible. Like in mice lacking three Pi transporter alleles, FGF23 levels were inappropriately normal in WT mice. L-BAIBA is only the third hormone known to cause renal Pi excretion. Ablation of Pi transporters in skeletal muscle is only the second known factor aside from exercise that stimulates L-BAIBA secretion from muscle. Although there are many phosphaturic medications, supplementation of L-BAIBA offers a number of benefits to patients with metabolic syndrome, phosphate concentration disorders, myopathy, and CKD stages 1-5, while most other phosphaturic medications have serious side effects.

Furthermore, ablation of the type III Pi importers Pit1 (Slc20a1) and Pit2 (Slc20a2) in skeletal muscle (DKO) results in a skeletal muscle myopathy, which is perinatally lethal. Mice with ablation of three or less transporter alleles show a gene-dose dependent reduction of running endurance, while different from Hyp—a murine model of X-linked hypophosphatemia (XLH)—grip strength is unaffected, suggesting that FGF23 excess contributes to the myopathy seen in XLH. At the tissue level there is reduced expression of the Pi-exporter Xenotropic and Polytropic Retrovirus Receptor 1 (Xpr1) in these mice.

The method of treating or preventing nephropathy includes administering a therapeutically effective amount of a composition containing L-BAIBA. In various embodiments, the nephropathy is CKD. There are well-accepted methods of measuring renal function, and the primary measure of renal function is the glomerular filtration rate (GFR), which is often estimated as creatinine clearance in serum and creatinine concentrations in urine. CKD can be defined as having a GFR of less than 60 mL/min for about three months or more. The stages of CKD, can be classified as follows:

Stage 1: Kidney damage with normal or increased GFR (>90 mL/min/1.73 m²),

Stage 2: Mild reduction in GFR (60-89 mL/min/1.73 m²),

Stage 3: Moderate reduction in GFR (30-59 mL/min/1.73 m²),

Stage 4: Severe reduction in GFR (15-29 mL/min/1.73 m²),

Stage 5: Kidney failure (GFR <15 mL/min/1.73 m² or dialysis).

In various embodiments, the method of treating nephropathy includes treating Stage 1 to Stage 5 CKD.

In various embodiments, the therapeutic methods described herein include a method of treating or preventing a phosphate concentration disease or disorder. The phosphate concentration disorder can be, in various embodiments, hyperphosphatemia (abnormally high phosphate). In various embodiments, a subject having hyperphosphatemia has a serum Pi concentration of at least or greater than about 4.5 mg/dL. In various embodiments, a subject having hyperphosphatemia has a serum Pi concentration of about 4.5 mg/dL to about 7 mg/dL. In various embodiments, administration of a therapeutically effective amount of L-BAIBA reduces the serum Pi concentration in a subject having hyperphosphatemia to at least or less than about 4.5 mg/dL.

L-BAIBA, but not D-BAIBA, is able to stimulate FGF23 synthesis in bone, resulting in unsuppressed FGF23 levels. This stimulation of FGF23 would be predicted to stimulate Pi excretion, particularly in FGF23-deficient forms of HFTC. L-BAIBA may also stimulate FGF23 in hyperphophaturic individuals and although counterproductive therapeutically, can help diagnostically to separate FGF23-dependent from -independent hypophosphatemic disorders. Blocking L-BAIBA in hypophosphatemias may improve hypophosphatemic control, if associated with elevated L-BAIBA levels. Conversely, blocking L-BAIBA in hyperphosphatemic conditions can improve FGF23 levels.

The Pi concentration disorder can be, in various embodiments, hypophosphatemia (abnormally low Pi). In various embodiments, a subject having hypophosphatemia has myopathy. In various embodiments, administering a therapeutically effective amount of L-BAIBA to a hypophosphatemic subject with myopathy improves or increases the muscular strength of the subject. Improvements in muscular strength can include improvement in grip strength, mobility, ability to bear weight, ability to lift weights, muscular flexibility, and the like.

The methods described herein comprise administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that raises or lowers serum Pi concentration.

In certain embodiments, administering the compound described herein to the subject allows for administering a lower dose of the additional therapeutic agent as compared to the dose of the additional therapeutic agent alone that is required to achieve similar results in treating or preventing Pi concentration disease or disorder in the subject. For example, in certain embodiments, the compound described herein enhances the Pi lowering or raising activity of the additional therapeutic compound, thereby allowing for a lower dose of the additional therapeutic compound to provide the same effect.

In certain embodiments, the compound described herein and the therapeutic agent are co-administered to the subject. In other embodiments, the compound described herein and the therapeutic agent are coformulated and co-administered to the subject. In certain embodiments, the subject is a mammal. In other embodiments, the mammal is a human. In yet other embodiments, the subject being submitted to any of the methods described herein is in need of being submitted to the method(s) in question.

### Combination Therapies

The compounds useful within the methods described herein may be used in combination with one or more additional therapeutic agents useful for treating a disease or disorder contemplated herein. These additional therapeutic agents may comprise compounds that are commercially available or synthetically accessible to those skilled in the art. These additional therapeutic agents are known to treat, prevent, or reduce the symptoms of nephropathy, hypophosphatemia, myopathy, hyperphosphatemia, and/or other diseases or disorders that result in abnormal levels of Pi in the serum.

In various embodiments, the additional therapeutic compound is selected from a Pit1 agonist, a Pit2 agonist, a Pit1 antagonist, a Pit2 antagonist, an L-valine supplement, L-valine deficient food, a 4-aminobutyrate aminotransaminase co-factor, a 4-aminobutyrate aminotransaminase inhibitor, vitamin D2 (ergocalciferol), vitamin D3 (cholecalciferol) 25-hydroxy vitamin D (calcidiol), 1,25-dihydroxy vitamin D (calcitriol), calcium acetate, sevelamer hydrochloride, sevelamer carbonate, iron sucrose, burosumab (Crysvita), and lanthanum carbonate.

In various embodiments, the additional therapeutic compound is selected from a Pit1 agonist, a Pit2 agonist, an L-valine supplement, and a 4-aminobutyrate aminotransaminase co-factor. In other embodiments, the additional therapeutic compound is a Pit1 antagonist, a Pit2 antagonist, L-valine deficient food, and a 4-aminobutyrate aminotransaminase inhibitor.

Administration of L-BAIBA and an additional therapeutic compound can, in various embodiments, result in a synergistic effect. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a disease or disorder contemplated herein. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated herein. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated herein. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms described herein are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated herein.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound described herein, alone or in combination with a second pharmaceutical agent; and instructions for using the compound to treat, prevent, or reduce one or more symptoms of a disease or disorder contemplated herein.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds described herein may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crospovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described hrein by pH-independent swelling.

Parenteral Administration

For parenteral administration, the compounds described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as lauryl, stearyl, or oleyl alcohols, or similar alcohol.

Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method described herein may be administered in the form of microparticles, for example, by injection or in the form of wafers or discs by implantation.

In one embodiment, the compounds described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of the disease or disorder in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds for use in the method described herein may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

### Examples

Various embodiments of the present invention can be better understood by reference to the following Examples which are offered by way of illustration. The present invention is not limited to the Examples given herein.

### Mice

Mouse lines are back-crossed to C57BL6 mice (Charles river laboratories strain ID 027) to reduce variation caused by genetic background and fed a standard chow containing 0.3% bio-available Pi (Teklad 2018S) or for selective experiments a high Pi (HPD diet, 1.2% bio-available Pi, Teklad TD85349). L-BAIBA can be supplied in the drinking water (see Chemical Reagents below). PCR-based genotyping can be done on each experimental animal using gDNA prepared by alkaline lysis from a tail- or ear-snip to verify presence of the Pit1 null, Pit2 null, HA-hPIT1^{tg/+} and HSA-Cre^{tg/+} alleles.

### Biochemical Measurements

All blood and urine studies for calcium, creatinine, phosphorus, iPTH, and c/iFGF23 can be performed using commercially available Stanbio kits, Immutopics and R&D ELISAs, or LC-MS/MS-based methods. The reagents are verified by the vendor and used within the expiration date stated on the package. Results can be analyzed using a Perkin Elmer 1420 Multilabel Counter Victor 3 currently located at the Anlyan Center, S-120 or using an Applied Biosystems API6500 QTrap interfaced to a Shimadzu HPLC (LC-20AD, SIL-20AC, CTO-20A) currently located in Anlyan Center, S-260.

### Cell Lines

The adenovirus packaging 293A cell line, murine myocyte cell line C2C12, or human RC13 and L6 cells can be used. These cell lines were authenticated by chromosomal analysis and/or short tandem repeat (STR) profiling. Absence of mycoplasma infection can be confirmed annually (or more frequently, if mycoplasma contamination is detected) using the e-Myco™ plus Mycoplasma PCR Detection Kit (#25237, JHSCIENCE, Maplewood, N.J.).

### Antibodies

Anti-HA tag Affinity Matrix New from rat IgG1 (Roche 11815016001), anti-V5-agarose (ab1229), anti-HA antibody (ab18181) and anti-V5 tag antibody (ab15828) to detect tagged versions of hPIT1 were used. Primary antibodies were validated using peptide used for immunization supplied by the vendor and/or sections from knockout mice. Immunization peptide and sections from knockout mice are expected to show background of the 1^{st} antibody. The 1^{st} antibody is also omitted and the 2^{nd} antibody is used alone, which determines background staining of the 2^{nd} antibody.

cDNAs

Shuttle vectors were obtained from PlasmID (Dana Faber Cancer Institute, Boston, Mass. and the Biodesign Institute at Arizona State University), which contain the full-length coding regions of human PIT1, PIT2 and XPR1 (HsCD00377128, XPR1-pENTR223; HsCD00377285, SLC20A1-pENTR223; HsCD00043621, SLC20A2-pDONR221). These vectors were sequence verified by the vendor and following subcloning into the adenoviral expression vector were Sanger sequenced once more to be ensure no point mutations are present.

### Chemical Reagents

Recombinant mouse FGF23 (6His-tagged Tyr25-Val251 [Arg179Gln]; 26.1 kDa) protein is resistant to furin-cleavage and thus more stable (R&D Systems). Bioactivity by induction of phosphaturia and hypophosphatemia 24 hrs. after intraperitoneal (40 ug/Kg) injection into mice. (S)-3-aminoisobutyric acid (L-BAIBA) (Adipogen Corp., San Diego, Calif.) can be provided at a concentration of 0.5 g/L in drinking water ad libitum, and the volume consumed per day monitored to assure equal intake between groups. Assuming consumption of 5 mL by a 25 g mouse, L-BAIBA intake will be 100 mg/kg/d.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### ENUMERATED EMBODIMENTS

The following exemplary embodiments are provided, the numbering of which is not to be construed as designating levels of importance:

Embodiment 1 provides a method of treating nephropathy, the method comprising administering to a subject in need thereof a therapeutically effective amount of L-BAIBA, or a salt, solvate, polymorph, prodrug, or N-oxide thereof.

Embodiment 2 provides the method of embodiment 1, wherein the nephropathy is selected from Alport syndrome, diabetic neuropathy, Fabry disease, focal segmental glomeronucleosis, glomerulonephritis, IgA nephropathy (Berger's disease), kidney stones, minimal change disease, nephrotic syndrome, polycystic kidney disease (PKD), and chronic kidney disease (CKD).

Embodiment 3 provides the method of any one of embodiments 1-2, wherein the method reduces serum phosphate (Pi) levels in the subject.

Embodiment 4 provides the method of any one of embodiments 1-3, wherein the serum Pi levels in the subject are reduced to at least about 4.5 mg/dL.

Embodiment 5 provides the method of any one of embodiments 1-4, wherein the composition comprises at least one additional therapeutic compound.

Embodiment 6 provides the method of any one of embodiments 1-5, wherein the one additional therapeutic compound is selected from a Pit1 agonist, a Pit2 agonist, a Pit1 antagonist, a Pit2 antagonist, an L-valine supplement, L-valine deficient food, a 4-aminobutyrate aminotransaminase co-factor, a 4-aminobutyrate aminotransaminase inhibitor, vitamin D2 (ergocalciferol), vitamin D3 (cholecalciferol) 25-hydroxy vitamin D (calcidiol), 1,25-dihydroxy vitamin D (calcitriol), calcium acetate, sevelamer hydrochloride, sevelamer carbonate, iron sucrose, burosumab, and lanthanum carbonate.

Embodiment 7 provides the method of any one of embodiments 1-6, wherein the composition is formulated for an administration route selected from oral, transdermal, transmucosal, (intra)nasal, (trans)rectal, intravesical, intrapulmonary, intraduodenal, intragastrical, intrathecal, subcutaneous, intramuscular, intradermal, intra-arterial, intravenous, intrabronchial, inhalation, and topical administration.

Embodiment 8 provides the method of any one of embodiments 1-7, wherein the subject is a mammal.

Embodiment 9 provides the method of any one of embodiments 1-8, wherein the mammal is a human.

Embodiment 10 provides a composition comprising L-BAIBA, or a salt, solvate, polymorph, prodrug, or N-oxide thereof, at least one additional therapeutic compound, and at least one pharmaceutically acceptable excipient.

Embodiment 11 provides the composition of embodiment 10, wherein the additional therapeutic compound is selected from a Pit1 agonist, a Pit2 agonist, a Pit1 antagonist, a Pit2 antagonist, an L-valine supplement, L-valine deficient food, a 4-aminobutyrate aminotransaminase co-factor, a 4-aminobutyrate aminotransaminase inhibitor, vitamin D2 (ergocalciferol), vitamin D3 (cholecalciferol) 25-hydroxy vitamin D (calcidiol), 1,25-dihydroxy vitamin D (calcitriol), calcium acetate, sevelamer hydrochloride, sevelamer carbonate, iron sucrose, burosumab, and lanthanum carbonate.

Embodiment 12 provides a method of treating a phosphate concentration disorder, the method comprising administering to a subject in need thereof a therapeutically effective amount of L-BAIBA or a salt, solvate, polymorph, prodrug, or N-oxide thereof.

Embodiment 13 provides the method of embodiment 12, wherein the phosphate concentration disorder is hyperphosphatemia.

Embodiment 14 provides the method of any one of embodiments 12-13, wherein the hyperphosphatemia is a result of CKD, metabolic acidosis, respiratory acidosis, familial hyperphosphatemic tumoral calcinosis (FHTC), rhabdomyolysis, and/or other conditions that result in abnormally high serum phosphate concentrations.

Embodiment 15 provides the method of any one of embodiments 12-14, wherein the hyperphosphatemia is a result of CKD.

Embodiment 16 provides the method of any one of embodiments 12-15, wherein the phosphate concentration disorder is hypophosphatemia.

Embodiment 17 provides the method of any one of embodiments 12-16, wherein the hypophosphatemia is a result of alcoholism, burns, starvation, diuretic use, primary hypoparathyroidism (PHPT), hereditary hypophosphatemic rickets with hypercalciuria (HHRH), X-linked hypophosphatemia (XLH), autosomal dominant hypophosphatemic rickets (ADHR), autosomal recessive hypophosphatemia (ARHP), tumor induced osteomalacia (TIO, also known as oncogenic osteomalacia), and/or other conditions that result in abnormally low serum phosphate concentrations.

Embodiment 18 provides the method of any one of embodiments 12-17, wherein the subject has myopathy, and wherein the myopathy is a result of hypophosphatemia.

Embodiment 19 provides the method of any one of embodiments 12-18, wherein the composition comprises at least one additional therapeutic compound.

Embodiment 20 provides the method of any one of embodiments 12-19, wherein the one additional therapeutic compound is selected from a Pit1 agonist, a Pit2 agonist, a Pit1 antagonist, a Pit2 antagonist, an L-valine supplement, L-valine deficient food, a 4-aminobutyrate aminotransaminase co-factor, a 4-aminobutyrate aminotransaminase inhibitor, vitamin D2 (ergocalciferol), vitamin D3 (cholecalciferol) 25-hydroxy vitamin D (calcidiol), 1,25-dihydroxy vitamin D (calcitriol), calcium acetate, sevelamer hydrochloride, sevelamer carbonate, iron sucrose, burosumab, and lanthanum carbonate.

Embodiment 21 provides the method of any one of embodiments 12-20, wherein the composition is formulated for an administration route selected from oral, transdermal, transmucosal, (intra)nasal, (trans)rectal, intravesical, intrapulmonary, intraduodenal, intragastrical, intrathecal, subcutaneous, intramuscular, intradermal, intra-arterial, intravenous, intrabronchial, inhalation, and topical administration.

1. A method of treating or ameliorating nephropathy in a subject, the method comprising:
administering to the subject a therapeutically effective amount of L-BAIBA, or a salt, solvate, polymorph, prodrug, or N-oxide thereof.

2. The method of claim 1, wherein the nephropathy is selected from Alport syndrome, diabetic neuropathy, Fabry disease, focal segmental glomeronucleosis, glomerulonephritis, IgA nephropathy (Berger's disease), kidney stones, minimal change disease, nephrotic syndrome, polycystic kidney disease (PKD), and chronic kidney disease (CKD), or any combination thereof.

3. The method of claim 1, wherein the method reduces serum phosphate (Pi) levels in the subject.

4. The method of claim 3, wherein the serum Pi levels in the subject are reduced to at least about 4.5 mg/dL.

5. The method of claim 1, wherein the composition comprises at least one additional therapeutic compound.

6. The method of claim 5, wherein the one additional therapeutic compound is selected from a Pit1 agonist, a Pit2 agonist, a Pit1 antagonist, a Pit2 antagonist, an L-valine supplement, L-valine deficient food, a 4-aminobutyrate aminotransaminase co-factor, a 4-aminobutyrate aminotransaminase inhibitor, vitamin D2 (ergocalciferol), vitamin D3 (cholecalciferol) 25-hydroxy vitamin D (calcidiol), 1,25-dihydroxy vitamin D (calcitriol), calcium acetate, sevelamer hydrochloride, sevelamer carbonate, iron sucrose, burosumab, and lanthanum carbonate, or any combination thereof.

7. The method of claim 1, wherein the composition is formulated for an administration route selected from oral, transdermal, transmucosal, (intra)nasal, (trans)rectal, intravesical, intrapulmonary, intraduodenal, intragastrical, intrathecal, subcutaneous, intramuscular, intradermal, intra-arterial, intravenous, intrabronchial, inhalation, or topical administration.

8. The method of claim 1, wherein the subject is a mammal.

9. The method of claim 8, wherein the mammal is a human.

10. A composition comprising:
L-BAIBA, or a salt, solvate, polymorph, prodrug, or N-oxide thereof,
at least one additional therapeutic compound, and
at least one pharmaceutically acceptable excipient.

11. The composition of claim 10, wherein the additional therapeutic compound is selected from a Pit1 agonist, a Pit2 agonist, a Pit1 antagonist, a Pit2 antagonist, an L-valine supplement, L-valine deficient food, a 4-aminobutyrate aminotransaminase co-factor, a 4-aminobutyrate aminotransaminase inhibitor, vitamin D2 (ergocalciferol), vitamin D3 (cholecalciferol) 25-hydroxy vitamin D (calcidiol), 1,25-dihydroxy vitamin D (calcitriol), calcium acetate, sevelamer hydrochloride, sevelamer carbonate, iron sucrose, burosumab, and lanthanum carbonate, or any combination thereof.

12. A method of treating or ameliorating a phosphate concentration disorder, the method comprising:
administering to a subject in need thereof a therapeutically effective amount of L-BAIBA, or a salt, solvate, polymorph, prodrug, or N-oxide thereof.

13. The method of claim 12, wherein the phosphate concentration disorder is hyperphosphatemia.

14. The method of claim 13, wherein the hyperphosphatemia is a result of CKD, metabolic acidosis, respiratory acidosis, familial hyperphosphatemic tumoral calcinosis (FHTC), rhabdomyolysis, or other conditions that result in abnormally high serum phosphate concentrations.

15. The method of claim 13, wherein the hyperphosphatemia is a result of CKD.

16. The method of claim 12, wherein the phosphate concentration disorder is hypophosphatemia.

17. The method of claim 16, wherein the hypophosphatemia is a result of alcoholism, burns, starvation, diuretic use, primary hypoparathyroidism (PHPT), hereditary hypophosphatemic rickets with hypercalciuria (HHRH), X-linked hypophosphatemia (XLH), autosomal dominant hypophosphatemic rickets (ADHR), autosomal recessive hypophosphatemia (ARHP), tumor induced osteomalacia (TIO, also known as oncogenic osteomalacia), or other conditions that result in abnormally low serum phosphate concentrations.

18. The method of claim 16, wherein the subject has myopathy, and wherein the myopathy is a result of hypophosphatemia.

19. The method of claim 12, wherein the composition comprises at least one additional therapeutic compound.

20. The method of claim 19, wherein the one additional therapeutic compound is selected from a Pit1 agonist, a Pit2 agonist, a Pit1 antagonist, a Pit2 antagonist, an L-valine supplement, L-valine deficient food, a 4-aminobutyrate aminotransaminase co-factor, a 4-aminobutyrate aminotransaminase inhibitor, vitamin D2 (ergocalciferol), vitamin D3 (cholecalciferol) 25-hydroxy vitamin D (calcidiol), 1,25-dihydroxy vitamin D (calcitriol), calcium acetate, sevelamer hydrochloride, sevelamer carbonate, iron sucrose, burosumab, and lanthanum carbonate, or any combination thereof.

21. The method of claim 12, wherein the composition is formulated for an administration route selected from oral, transdermal, transmucosal, (intra)nasal, (trans)rectal, intravesical, intrapulmonary, intraduodenal, intragastrical, intrathecal, subcutaneous, intramuscular, intradermal, intra-arterial, intravenous, intrabronchial, inhalation, or topical administration.
